(19) United States
(12) Patent Application Publication
(10) Pub. No.: US 2022/0062050 A1
(43) Pub. Date: Mar. 3, 2022

(54) PERIOPERATIVE EYE PATCH

(71) Applicant: Linda L. Zeineh, M.D., Inc., Orange, CA (US)

(72) Inventor: Linda Zeineh, Fullerton, CA (US)

(73) Assignee: Linda L. Zeineh, M.D., Inc., Orange, CA (US)

(21) Appl. No.: 17/011,865

(22) Filed: Sep. 3, 2020

(51) Int. Cl. A61F 9/04

(52) U.S. Cl. CPC A61F 9/04

(57) ABSTRACT

A disposable perioperative eye patch for protecting the eyes and eyelashes of anesthetized patients during and immediately after surgery. The perioperative eye patch may be applied to the eyes of an anesthetized patient prior to surgery to securely, yet delicately, keep a patient's eyes closed during surgery to avoid ocular dryness and injury. The perioperative eye patch may be comprised of a dressing, a cushion, and a cover, allowing the perioperative eye patch to be securely attached to a patient's periorbital region. The size of the dressing, as well as the adhesive used, allow the perioperative eye patch to be applied and removed from a patient's periorbital region without causing damage to a patient's eyelashes during the perioperative period.

### FIELD OF USE

The present disclosure relates generally to the field of perioperative eye protection, and more specifically to a disposable perioperative eye patch that can be applied to and removed from an anesthetized patient's periorbital area without causing damage to the patient's eyelashes.

### BACKGROUND

Anesthetized patients must frequently have their eyelids closed during surgery. This is because an anesthetized patient's eyes will otherwise dry out due to the unconscious patient not being able to blink, causing irritation and potentially serious ocular injury. Alternatively, surgeons and attending medical staff may opt to regularly administer eye drops to the patient's eyes during surgery to keep the patient's eyes moisturized.

Current methods for keeping patients' eyes closed during surgery are unsatisfactory for several reasons. First, there is no disposable, single use, prepackaged, convenient, optimal, and sterile product that can be readily applied to fasten patients' eyelids closed. Currently, operating room personnel will often cut strips of gauze to roughly appropriate sizes, and then secure the hand-cut gauze patches to a patient's eye area with medical tape or another adhesive. The lack of products specifically geared and sized toward protecting an anesthetized patient's eyes during surgery costs anesthesiologists and surgeons valuable time, requiring them to improvise eye covers of their own. Besides the cost of surgical time, such improvised eyelid covers may be ineffective due to being improperly sized.

Additionally, existing methods for taping patients' eyelids closed often cause damage to patient eyelashes when the tape or gauze is removed post-surgery. This is because the adhesives used to secure patches onto the patient's eyes easily stick to a patient's eyelashes, and may inadvertently remove eyelashes when the eye patch is removed post-surgery. This problem is exacerbated when surgical patients have false eyelashes, which are frequently longer and thicker than natural eyelashes, allowing greater surface area interaction with the adhesive tape. False eyelashes may also be less securely attached to the patient's skin compared to natural eyelashes, making them more susceptible to inadvertent post-surgery removal.

This represents an important financial interest for many surgical patients, who often pay a significant amount of money for obtaining and maintaining false eyelashes. The US false eyelash market was approximately $300 million in 2016, and has been increasing every year since then. Due to the expensive nature of having and maintaining false eyelashes, patients frequently discuss in the preop area that they want their eyelashes to be preserved during surgery. It is therefore desirable to find a way to protect surgery patients' false eyelashes from perioperative damage, by using improved and more sensitive eye protection measures.

Thus, there is a need for a sterile, easy-to-use, prepackaged perioperative eye protection solution that can protect a user's eyes from drying out during surgery, while also being delicate and easily removable in order to avoid damaging a user's eyelashes when removed after surgery.

### SUMMARY

To minimize the limitations in the cited references, and to minimize other limitations that will become apparent upon reading and understanding the present specification, the perioperative eye patch disclosed herein discloses a perioperative eye patch that may be used to delicately keep a patient's eyes closed during surgery. In addition, the eye patch of the present disclosure may be easily removed after surgery in a manner that avoids causing damage to the patient's eyelashes.

One embodiment may be a perioperative eye patch, comprising a dressing, a cushion, and a cover. In one embodiment, the dressing may be a sheet made of a woven fabric or plastic (such as PVC or polyethylene). The dressing may be further comprised of an adhesive face and a non-adhesive face. The dressing's adhesive face may be coated with an adhesive, such as methacrylates, vinyl resins, gels, or other common bandage adhesives. The cushion may be comprised of cotton, felt, gel, foam, or gauze, and may preferably be coated in a fluid resistant material.

In one embodiment, the cushion may be secured to the adhesive face of the dressing via a glue or other appropriate adhesive. In one embodiment, the cushion may be secured to the center of the adhesive face, such that a perimeter of the adhesive face remains exposed.

In some embodiments, the cushion may be replaced with a non-adhesive dressing, such as a non-absorbent fabric or material that is bonded on both sides with a perforated non-adherent film. The purpose of the cushion is not to absorb fluids, but to engage with a wearer's eyelids and keep them closed during surgery.

In one embodiment, the cover may be a thin, transparent sheet, such as a plastic. The cover may be lightly attached to the adhesive face of the dressing by physical contact with the adhesive face. In some embodiments, the cover may be peeled off to expose the dressing and cushion underneath, allowing the perioperative eye patch to be applied to a patient's periorbital area.

In some embodiments, the perioperative eye patch may further comprise a packaging envelope. Preferably, the packaging envelope allows the perioperative eye patch to be individually packaged in sterile, standard dressing operating room packaging. The use of sterile packaging provides for an easy-to-apply perioperative eye patch that saves valuable time in the operating room prior to surgery.

In one embodiment, the dressing and the cushion may be appropriately sized to cover a patient's periorbital area. In one embodiment, the dressing may be oval in shape, with a height and width of approximately 5 cm×7 cm, respectively. In one embodiment, the cushion may be attached to the center of the adhesive face of the dressing. The cushion, in some embodiments, may be oval in shape, with a height and width of approximately 3 cm×5 cm, respectively. The height and width dimensions provided herein for the dressing and cushion are provided by way of example and not limitation. The dimensions of the dressing and the cushion may be increased or reduced as appropriate based on the facial features of a particular patient.

Preferably, the perioperative eye patch of the present disclosure is large enough that, when applied to a patient's periorbital area, the adhesive face of the dressing avoids any contact with a patient's eyelashes. Rather, only the cushion (attached to the adhesive face of the dressing) directly contacts a patient's eyelids and eyelashes. The adhesive face of the dressing only contacts the patient's skin and/or eyebrows in the periorbital area surrounding the patient's eye and eyelashes.

The perioperative eye patch preferably: is flush with the eye orbit when in use; is made of waterproof material, so that the preparation and fluids used in the surgical theatre will be deflected and it will therefore better adhere to underlying skin along the perimeter; is non-absorbent, so that fluids in the theatre field will not be absorbed; has a thick central cushion (0.5 cm to 2 cm) in order to engage with the eyelid of the patient and protect their eyelashes.

In one embodiment, the perioperative eye patch may comprise a dressing, a cushion, and a cover. The dressing may further comprise an adhesive face and a non-adhesive face. The cushion may be attached to the center of the dressing's adhesive face, such that an adhesive perimeter is created on the adhesive face around the cushion. The cover may be configured to removeably attach to the dressing's adhesive face, such that when the cover is removed from the adhesive face, the cushion and the adhesive face are configured to engage with a periorbital region of a surgical patient's eye. Preferably, the cushion engages with an eyelid and a plurality of eyelashes of the surgical patient's eye, and keeps the eyelid closed while protecting the plurality of eyelashes from being damaged. Removing the cover from the adhesive face may preferably result in the adhesive perimeter being configured to removeably adhere to an outer rim of the periorbital region of the surgical patient's eye. The perioperative eye patch may be configured to be used once and discarded thereafter. The dressing and cushion may be each substantially oval in shape, while the cushion may have a thickness in the range of approximately 0.5 cm to 2 cm. The cover may be a thin sheet of plastic. The dressing may be made of a woven fabric; a non-woven fabric; plastic; polyvinyl chloride; or polyethylene. The dressing may be coated in plastic, while the adhesive perimeter may be coated with an adhesive selected from the group of adhesives consisting of one or more of: methacrylates; vinyl resins; gels; and combinations thereof. The cushion may be comprised of a material selected from the group of materials consisting of one or more of: felt, foam, gel, and combinations thereof. The cushion and the dressing may be substantially water resistant. In some embodiments, the disposable perioperative eye patch may further comprise a single use sterile package that encloses and keeps sterile until use the dressing, cushion, and cover. Preferably, the package may be substantially water resistant.

In one embodiment, the perioperative eye patch may comprise a dressing, a cushion, and a cover. The dressing may further comprise an adhesive face and a non-adhesive face. The cushion may be attached to the center of the dressing's adhesive face, such that an adhesive perimeter is created on the adhesive face around the cushion. The cover may be configured to removeably attach to the dressing's adhesive face, such that when the cover is removed from the adhesive face, the cushion and the adhesive face are configured to engage with a periorbital region of a surgical patient's eye. Preferably, the cushion engages with an eyelid and a plurality of eyelashes of the surgical patient's eye, and keeps the eyelid closed while protecting the plurality of eyelashes from being damaged. Removing the cover from the adhesive face may preferably result in the adhesive perimeter being configured to removeably adhere to an outer rim of the periorbital region of the surgical patient's eye. The perioperative eye patch may be configured to be used once and discarded thereafter. The dressing and cushion may be each substantially oval in shape, while the cushion may have a thickness in the range of approximately 0.5 cm to 2 cm. The cushion may be comprised of a material selected from the group of materials consisting of one or more of: felt, foam, gel, and combinations thereof. The cushion may be substantially water resistant. The disposable perioperative eye patch may further comprise a single use water resistant sterile package that encloses and keep sterile until use the dressing, cushion, and cover.

Other features and advantages inherent in the perioperative eye patch of the present disclosure, besides those which are claimed and disclosed, will become apparent to those skilled in the art from the following detailed description and its accompanying drawings.

### BRIEF DESCRIPTION OF THE DRAWINGS

The drawings are of illustrative embodiments. They do not illustrate all embodiments. Other embodiments may be used in addition or instead. Details which may be apparent or unnecessary may be omitted to save space or for more effective illustration. Some embodiments may be practiced with additional components or steps and/or without all of the components or steps, which are illustrated. When the same numeral appears in different drawings, it refers to the same or like components or steps.

FIG. 1 is an illustration of a front perspective view of one embodiment of the perioperative eye patch.

FIG. 2 is an illustration of a front perspective view of a second embodiment of the perioperative eye patch.

FIG. 3 is an illustration of a back view of one embodiment of the perioperative eye patch.

FIG. 4 is an illustration of an exploded view of one embodiment of the perioperative eye patch.

FIG. 5 is an illustration of a side view of one embodiment of the perioperative eye patch.

FIG. 6 is an illustration of one embodiment of the perioperative eye patch affixed to a patient.

FIG. 7 is an illustration of one embodiment of a perioperative eye patch in a single use package.

### DETAILED DESCRIPTION OF THE ILLUSTRATIVE EMBODIMENTS

In the following detailed description, numerous specific details are set forth in order to provide a thorough understanding of various aspects of one or more embodiments. However, these embodiments may be practiced without some or all of these specific details. In other instances, well-known methods, procedures, and/or components have not been described in detail so as not to unnecessarily obscure aspects of embodiments.

While multiple embodiments are disclosed, still other embodiments will become apparent to those skilled in the art from the following detailed description. As will be realized, these embodiments are capable of modifications in various obvious aspects, all without departing from the spirit and scope of protection. Accordingly, figures and the detailed descriptions thereof are to be regarded as illustrative in nature and not restrictive. Also, the reference or non-reference to a particular embodiment shall not be interpreted to limit the scope of protection.

In the following description, certain terminology is used to describe certain features of one or more embodiments. For purposes of the specification, unless otherwise specified, the term “substantially” refers to the complete or nearly complete extent or degree of an action, characteristic, property, state, structure, item, or result. For example, in one embodiment, an object that is “substantially” located within a housing would mean that the object is either completely within a housing or nearly completely within a housing. The exact allowable degree of deviation from absolute completeness may in some cases depend on the specific context. However, generally speaking, the nearness of completion will be so as to have the same overall result as if absolute and total completion were obtained. The use of “substantially” is also equally applicable when used in a negative connotation to refer to the complete or near complete lack of an action, characteristic, property, state, structure, item, or result.

As used herein, the terms “approximately” and “about” generally refer to a deviance of within 5% of the indicated number or range of numbers. In one embodiment, the terms “approximately” and “about,” may refer to a deviance of between 0.0001-10% from the indicated number or range of numbers.

The present specification discloses a perioperative eye patch designed to minimize damage to an anesthetized patient's eyelashes during and immediately after surgery.

Disclosed are components that may be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all embodiments of this application including, but not limited to, steps in disclosed methods. Thus, if there are a variety of additional steps that may be performed it is understood that each of these additional steps may be performed with any specific embodiment or combination of embodiments of the disclosed methods.

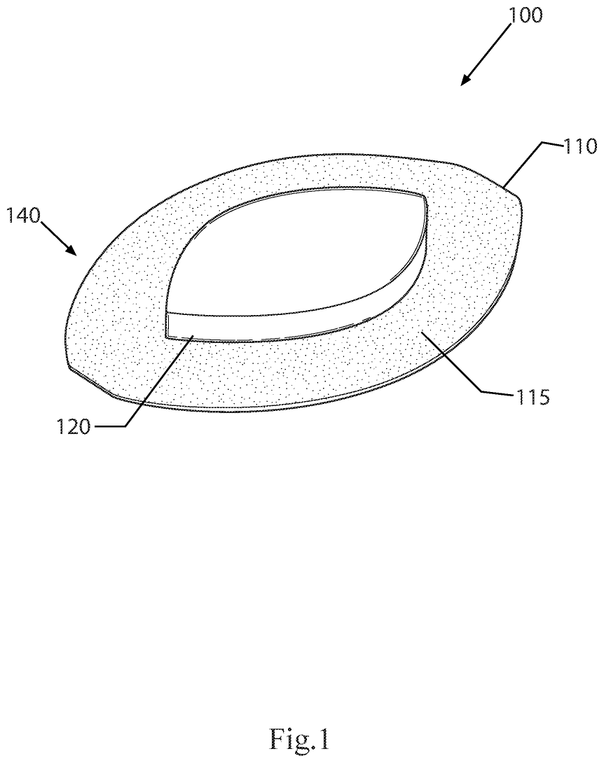

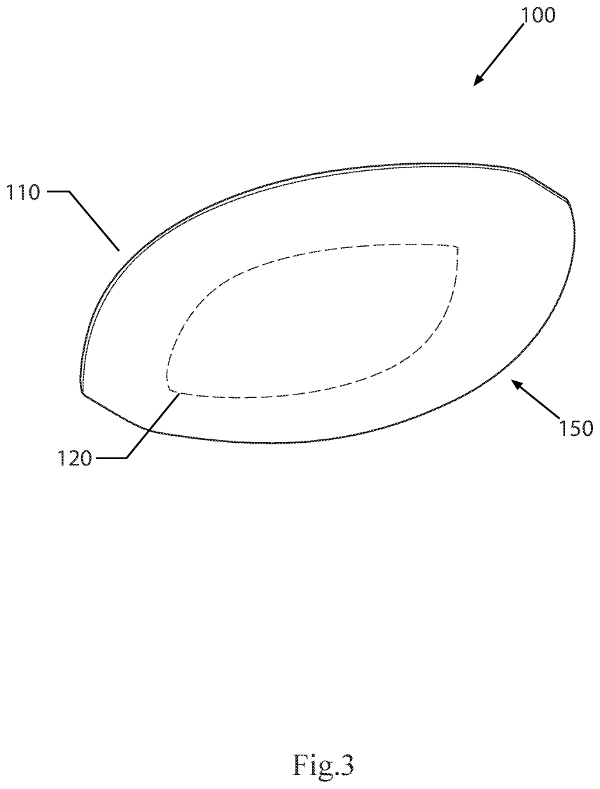

FIG. 1 is an illustration of a front perspective view of one embodiment of the perioperative eye patch. As shown in FIG. 1, the perioperative eye patch 100 (or pad) may comprise a dressing 110 and a cushion 120. The dressing 110 may further comprise an adhesive face 140 on the front side, and a non-adhesive face 150 on the back side (shown in FIG. 3). The cushion 120 may preferably be attached to the center of the adhesive face 140, such that an adhesive perimeter 115 remains exposed at the outer edges of the adhesive face 140. The adhesive perimeter 115 may be coated with a common bandage adhesive, such as methcrylates, vinyl resins, or gels, to allow the perioperative eye patch 100 to be both firmly attached, but easily removed from a patient's skin after application. By contrast, the cushion 120 may preferably be adhered firmly to the adhesive face 140 with a stronger glue or adhesive, to ensure that the cushion 120 remains attached to the adhesive face 140 of the dressing 110. The dressing 110 may be the main backing of the pad 100.

In some embodiments, the cushion 120 may be a non-woven cotton pad. In other embodiments, the cushion 120 may be a non-adhesive dressing, such as an absorbent cotton fabric bonded on one or both sides with a perforated non-adherent film. The cushion 120 may be comprised of cotton, felt, gel, foam, or gauze, and may preferably be coated in a fluid resistant material. Because the primary purpose of the eye patch 100 is to hold the wearer's eyelid closed during surgery, the cushion does not need to be absorbent and it is preferably not. When made from liquid resistant materials, the cushion 120 and dressing 110 do not absorb the liquids that may contact the eye patch 100 during surgery. Although the patch 100 is preferably oval in shape as shown, it may be any shape or size that is configured to cover the eye of the wearer.

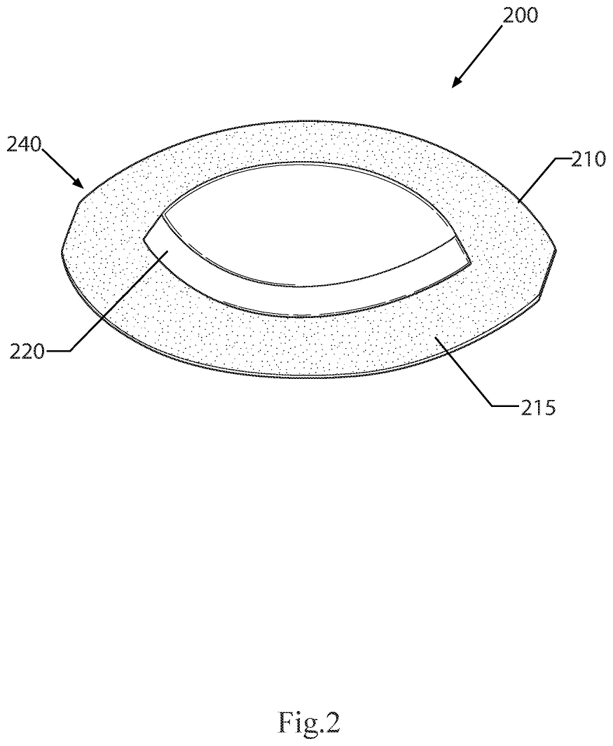

FIG. 2 is an illustration of front perspective view of a second embodiment of the perioperative eye patch. As shown in FIG. 2, the eye patch 200 may have an adhesive front face 240 and may comprise a dressing 210 and a cushion 220. The adhesive front face 240 may have an adhesive perimeter 215 that is formed around the cushion 220. FIG. 2 shows that the cushion 220 may be tapered. The cushion 220 may be any shape that allows the patch 200 to keep the patient's eye closed.

FIG. 3 is an illustration of a back view of one embodiment of the perioperative eye patch. As shown in FIG. 3, the back side of the dressing 110 may comprise a non-adhesive face 150. The dressing 110 is, preferably, only adhesive on its front face, which is the side that engages with the patient's face. The back side portion of the dressing 110 is preferably not coated with an adhesive. FIG. 3 shows where the cushion is on the front side of the dressing 110.

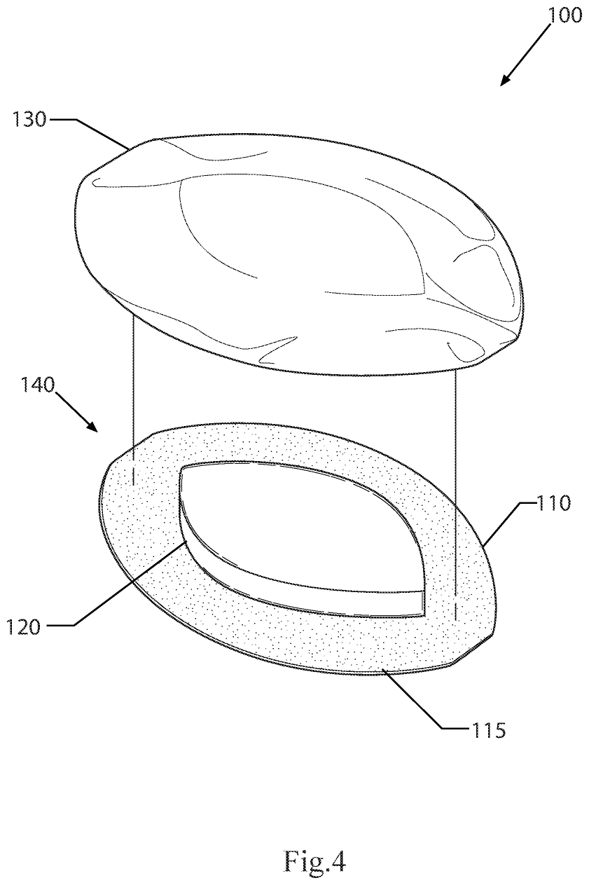

FIG. 4 is an illustration of an exploded view of one embodiment of the perioperative eye patch. As shown in FIG. 4, the cover 130 may be peeled off and removed, exposing the adhesive perimeter 115 and the cushion 120, such that the perioperative eye patch 100 may be applied to a patient's periorbital area. Preferably, the cover 130 may be made of a plastic, wax, or be coated as such so that it is easily removeable from the adhesive perimeter 115. The cover 130 serves to keep the adhesive face 140 and the cushion 120 clean and sanitary until the perioperative eye patch 100 is ready to be applied to the patient. The cover 130 mildly adheres to the adhesive perimeter 115, such that the cover 130 forms a sanitary barrier over the entire front face of the perioperative eye patch 100. Once the cover 130, which may be one, two, or more separate pieces, has been peeled off and removed, it can be discarded. With the adhesive perimeter 115 now exposed, the perioperative eye patch 100 is ready to be applied.

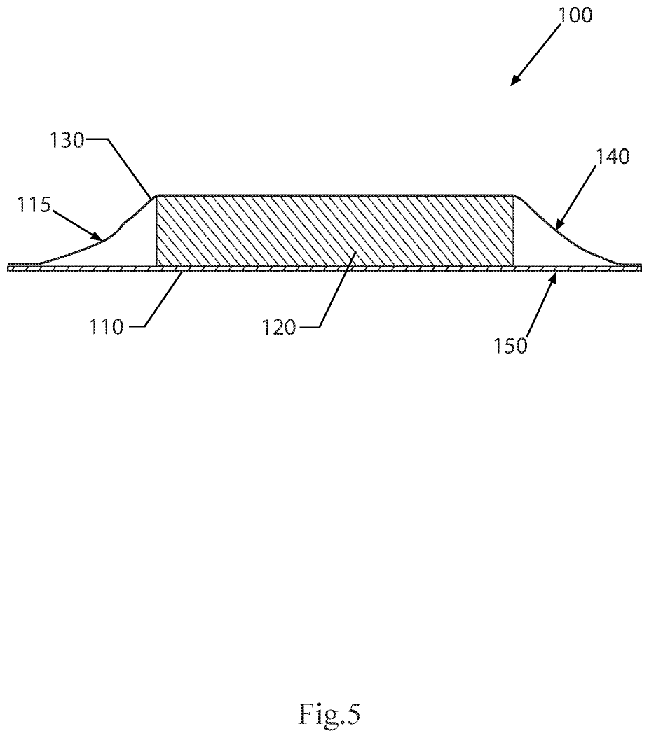

FIG. 5 is an illustration of a side view of one embodiment of the perioperative eye patch. As shown in FIG. 5, the cushion 120 is attached to the center of the adhesive face 140 of the dressing 110. The cover 130 may be attached to the adhesive perimeter 115, such that the cushion 120 is sandwiched between the plastic cover 130 and the dressing 110. The dressing 110 may have a non-adhesive face 150. As shown, the cushion 120 may have a portion that is at least 0.5 cm thick, but is preferably not more than 2 cm thick.

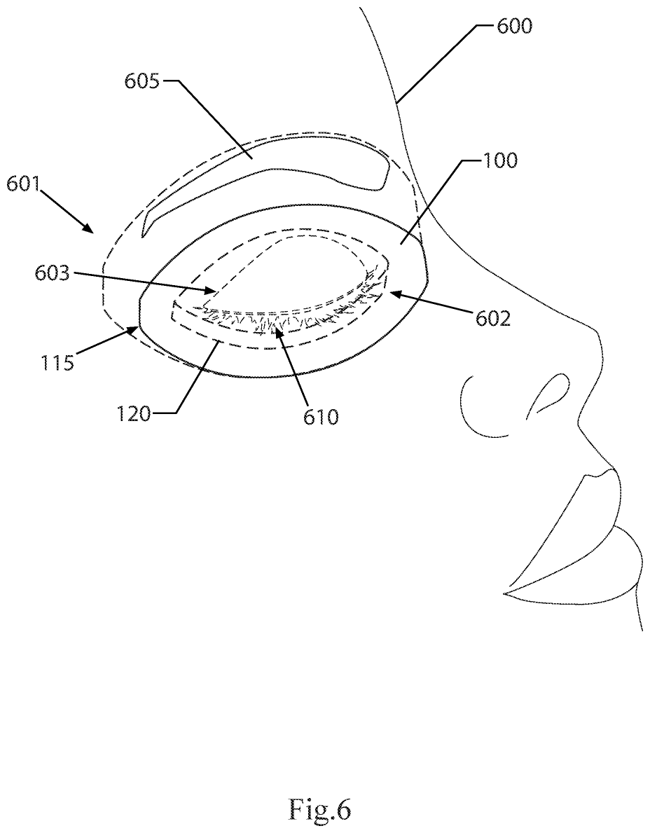

FIG. 6 is an illustration of one embodiment of the perioperative eye patch affixed to a patient. As shown in FIG. 6, the perioperative eye patch 100 may be applied to the periorbital area 601 of a patient 600. The size and shape of the periorbital eye patch 100 allows it to cover a patient's periorbital area 601, including the patient's eyes 602, eyelashes 610, and eyelids 603. The perioperative eye patch 100 gently provides sufficient pressure on a patient's eyelids 603 to ensure that they remain closed while the perioperative eye patch 100 is applied. By so doing, the patient's eye 602 remains protected and will not dry out during surgery. The thickness and delicateness of the cushion 120 additionally ensure that the patient's eyelashes 610 are not damaged during application via excessive pressure, adhesion, or tugging.

Additionally, the size of the perioperative eye patch 100 is large enough to ensure that the adhesive perimeter 115 of the adhesive layer 110 avoids contact with the patient's eyelashes 610. In this manner, the patient's eyelashes 610 will not be damaged or inadvertently removed when the perioperative eye patch 100 is removed after surgery. Preferably, the only physical contact that the patient's eyelashes 610 have with the perioperative eye patch 100 is with the cushion 120. The cushion 120 may preferably be made of felt, gel, foam, cotton, or a similarly delicate, non-adhesive material, such that it will avoid causing damage to the patient's eyelashes 610. In some embodiments, the cushion may be so large that it covers the eyebrows 605 as well, but the most important feature is to safely guard the eyelashes 610.

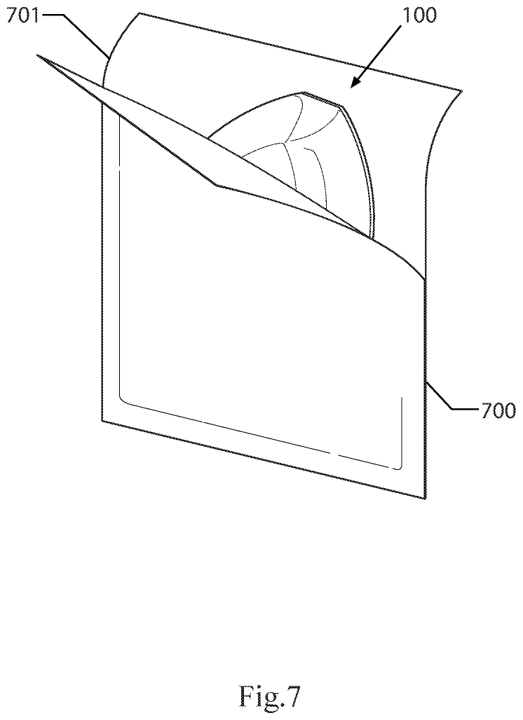

FIG. 7 is an illustration of one embodiment of a perioperative eye patch in a single use package. As shown in FIG. 7, the perioperative eye patch 100 may be contained within a single-use, sterile sanitary package 700. The eye patch 100 may be accessed by peeling (or other ways of opening, such as cutting or tearing) the package 700 open. In various embodiments, package may be pre-notched or perforated, or be able to be peeled apart 701.

Unless otherwise stated, all measurements, values, ratings, positions, magnitudes, sizes, locations, and other specifications, which set forth in this specification, including in the claims that follow, are approximate, not exact. They are intended to have a reasonable range, which is consistent with the functions to which they relate and with what is customary in the art to which they pertain.

The foregoing description of the preferred embodiment has been presented for the purposes of illustration and description. While multiple embodiments are disclosed, still other embodiments will become apparent to those skilled in the art from the above detailed description, which shows and describes the illustrative embodiments. As will be realized, these embodiments are capable of modifications in various obvious aspects, all without departing from the spirit and scope of the present disclosure. Accordingly, the detailed description is to be regarded as illustrative in nature and not restrictive. Also, although not explicitly recited, one or more additional embodiments may be practiced in combination or conjunction with one another. Furthermore, the reference or non-reference to a particular embodiment shall not be interpreted to limit the scope of protection. It is intended that the scope of protection not be limited by this detailed description, but by the claims and the equivalents to the claims that are appended hereto.

Except as stated immediately above, nothing which has been stated or illustrated is intended or should be interpreted to cause a dedication of any component, step, feature, object, benefit, advantage, or equivalent to the public, regardless of whether it is or is not recited in the claims.

What is claimed is:

1. A disposable perioperative eye patch, comprising:
a dressing;
a cushion; and
a cover;
wherein said dressing comprises an adhesive face and a non-adhesive face;
wherein said cushion is attached to said adhesive face such that an adhesive perimeter on said adhesive face is created around said cushion;
wherein said cover is configured to removeably attach to said adhesive face;
wherein when said cover is removed from said adhesive face, said cushion and said adhesive face of said dressing are configured to engage with a periorbital region of a surgical patient's eye, such that said cushion engages with an eyelid and a plurality of eyelashes of said surgical patient's eye and keeps said eyelid closed and protects said plurality of eyelashes from being damaged;
wherein when said cover is removed from said adhesive face, said adhesive perimeter is configured to removeably adhere to an outer rim of said periorbital region of said surgical patient's eye; and
wherein said disposable perioperative eye patch is configured to be used once and discarded thereafter.

2. The disposable perioperative eye patch of claim 1, wherein said dressing and said cushion are each substantially oval in shape.

3. The disposable perioperative eye patch of claim 1, wherein said cushion has a thickness in the range of approximately 0.5 cm to 2 cm.

4. The disposable perioperative eye patch of claim 1, wherein said cover is a thin sheet of plastic.

5. The disposable perioperative eye patch of claim 1, wherein said dressing is made of a woven fabric.

6. The disposable perioperative eye patch of claim 1, wherein said dressing is made of a non-woven fabric.

7. The disposable perioperative eye patch of claim 1, wherein said dressing is made of plastic.

8. The disposable perioperative eye patch of claim 7, wherein said dressing is made of polyvinyl chloride or polyethylene.

9. The disposable perioperative eye patch of claim 1, wherein said dressing is coated in plastic.

10. The disposable perioperative eye patch of claim 4, wherein said adhesive perimeter is coated with an adhesive selected from the group of adhesives consisting of one or more of: methacrylates; vinyl resins; gels; and combinations thereof.

11. The disposable perioperative eye patch of claim 1, wherein said cushion is comprised of a material selected from the group of materials consisting of one or more of: felt, foam, gel, and combinations thereof.

12. The disposable perioperative eye patch of claim 1, wherein said cushion is substantially water resistant.

13. The disposable perioperative eye patch of claim 1, wherein said dressing is substantially water resistant.

14. The disposable perioperative eye patch of claim 1, further comprising a single use sterile package that encloses and keeps sterile until use said dressing, said cushion, and said cover.

15. The disposable perioperative eye patch of claim 14, wherein said package is substantially water resistant.

16. A disposable perioperative eye patch, comprising:
a dressing;
a cushion; and
a cover;
wherein said dressing comprises an adhesive face and a non-adhesive face;
wherein said cushion is attached to said adhesive face such that an adhesive perimeter on said adhesive face is created around said cushion;
wherein said cover is configured to removeably attach to said adhesive face;
wherein when said cover is removed from said adhesive face, said cushion and adhesive face of said dressing is configured to engage with a periorbital region of a surgical patient's eye, such that said cushion engages with an eyelid and a plurality of eyelashes of said surgical patient's eye and keeps said eyelid closed and protects said plurality of eyelashes from being damages;
wherein when said cover is removed from said adhesive face, said adhesive perimeter is configured to removeably adhere to an outer rim of said periorbital region of said surgical patient's eye;
wherein said disposable perioperative eye patch is configured to be used once and discarded thereafter;
wherein said dressing and said cushion are each substantially oval in shape; and
wherein said cushion has a thickness in the range of approximately 0.5 cm to 2 cm.

17. The perioperative eye patch of claim 16, wherein said cushion is comprises a material selected from the group of materials consisting of one or more of: felt, foam, gel, and combinations thereof.

18. The perioperative eye patch of claim 17, wherein said dressing is substantially water resistant.

19. The perioperative eye patch of claim 1, further comprising a single use water resistant sterile package that encloses and keeps sterile until use said dressing, said cushion, and said cover.

20. A disposable perioperative eye patch, comprising:
a dressing;
a cushion;
a cover; and
a single use sterile package that encloses and keeps sterile until use said dressing, said cushion, and said cover;
wherein said dressing comprises an adhesive face and a non-adhesive face;
wherein said cushion is attached to said adhesive face such that an adhesive perimeter on said adhesive face is created around said cushion;
wherein said cover is configured to removeably attach to said adhesive face;
wherein when said cover is removed from said adhesive face, said cushion and adhesive face of said dressing is configured to engage with a periorbital region of a surgical patient's eye, such that said cushion engages with an eyelid and a plurality of eyelashes of said surgical patient's eye and keeps said eyelid closed and protects said plurality of eyelashes from being damages;
wherein when said cover is removed from said adhesive face, said adhesive perimeter is configured to removeably adhere to an outer rim of said periorbital region of said surgical patient's eye; and
wherein said disposable perioperative eye patch is configured to be used once and discarded thereafter;
wherein said dressing and said cushion are each substantially oval in shape;
wherein said cushion has a thickness in the range of approximately 0.5 cm to 2 cm;
wherein said cover is a thin sheet of plastic;
wherein said adhesive perimeter is coated with an adhesive selected from the group of adhesives consisting of one or more of: methacrylates; vinyl resins; gels; and combinations thereof;
wherein said cushion is comprises a material selected from the group of materials consisting of one or more of: felt, foam, gel, and combinations thereof;
wherein said dressing is substantially water resistant; and
wherein said package is substantially water resistant.
